(19) United States
(12) Patent Application Publication
(10) Pub. No.: US 2026/0207875 A1
(43) Pub. Date: Jul. 23, 2026

(54) Body-mind Management System Based on Drama Field, Voiceprint and AI Technology

(71) Applicant: Southern Medical University, Guangzhou (CN)

(72) Inventor: Xiang ZHANG, Guangzhou (CN)

(73) Assignee: Southern Medical University, Guangzhou (CN)

(21) Appl. No.: 19/183,726

(22) Filed: Apr. 18, 2025

(30) Foreign Application Priority Data
Jan. 20, 2025 (CN) 202510083983.8

(51) Int. Cl. A61M 21/00; A61B 5/00; A61B 5/16; G06F 3/01; G10L 15/04; G10L 25/63

(52) U.S. Cl. CPC A61M 21/00; A61B 5/165; A61B 5/4803; A61B 5/4836; A61B 5/4854; A61B 5/7264; G06F 3/015; G10L 15/04; G10L 25/63; A61M 2021/0027; G06F 2203/011

(57) ABSTRACT

A body-mind management system based on drama field, voiceprint and AI technologies, comprising a data acquisition module, a modelling module for building a Huiyin agent, a Yunjing knowledge base and a sound-assisted model, a programming module for building a personalized body-mind management program for the user, an analysis and evaluation module for analyzing the human voice audios uploaded by the user and monitoring and evaluating the user's body-mind state, and a field building module for building specific environments under different drama fields. By selecting sound materials in natural environment, mainly natural sound sources, natural voiceprints are used greatly to build a natural ecological field more suitable for human beings, so that people in cities can get closer to nature, open their sensory systems, relax themselves, balance emotions, stimulate potential, and improve comprehensive field efficiency.

### RELATED APPLICATIONS

The present application claims the benefit of priority of Chinese Patent Application No. 202510083983.8, filed Jan. 20, 2025, and entitled “Body-mind Management System Based on Drama Field, Voiceprint and AI Technology,” the entire content of which is incorporated herein by reference.

### TECHNICAL FIELD

The present disclosure relates to a technical field of physical and mental health, in particular to a body-mind management system based on drama field, voiceprint and AI technologies.

### BACKGROUND

Based on the five elements basic theory of traditional Chinese medicine, the voiceprint five elements AI system will combine acoustic and AI technologies, and can analyze the body-mind state of individuals through human voice, and use the sound materials collected in natural ecology in a compatible manner to achieve the purpose of stimulating cognitive potential and adjusting emotional state. Based on this integrated technology, we can further organically combine elements such as sound, image, light, season into a specific space to immerse people in it, and make them actively adjust their body-mind states in the process of fully interacting with the environmental energy field by means of dramatic interpretation.

Although some existing body-mind management systems can realize a certain degree of body-mind management, the sound materials used in them are single and mostly artificially synthesized, so there is a phenomenon that the effect of body-mind management is not good.

To sum up, the sound materials used in some existing body-mind management systems are single and mostly artificially synthesized, and the problem of poor body-mind management effect has become an urgent problem to be solved in this field. Therefore, it is necessary to propose a body-mind management system based on drama field, voiceprint and AI technologies.

### SUMMARY

In order to solve the above problems, the present disclosure provides a body-mind management system based on drama field, voiceprint and AI technologies. By selecting sound materials in the natural environment, mainly natural sound sources, natural voiceprints are used greatly to build a natural ecological field more suitable for human beings, so that people who have lived in cities for a long time can get closer to nature, open their sensory systems, relax themselves, balance emotions, stimulate potential, and improve comprehensive field efficiency.

In order to achieve the above purpose, the technical solution of the present disclosure is as follows: a body-mind management system based on drama field, voiceprint and AI technologies, comprising the following modules:
a data acquisition module, configured to sample sounds in the natural environment and convert them into digital signals;
a modelling module, configured to build a Huiyin agent based on the digital signals, segment the human voice audios uploaded by the user and assign tag data through the Huiyin agent, establish a Yunjing knowledge base based on the tag data, and manage the human voice audios;
the modelling module further builds a sound-assisted model based on the Shenglan large-scale model;
a programming module, configured to classify the collected sounds in the natural environment based on the five-element theory of traditional Chinese medicine and the sound-assisted model, analyze the emotions and brain waves of the user to obtain the specific situation of the user, perform permutation and combination of sounds according to the specific situation of the user, and build a personalized body-mind management program for the user;
an analysis and evaluation module, configured to analyze the human voice audios uploaded by the user to obtain current body-mind state indexes of the user, and monitor and evaluate the physiological and psychological states of the user through AI technology to obtain the evaluation result; and
a field building module, configured to build a specific drama field based on the evaluation result obtained by the analysis and evaluation module.

Further, the sound-assisted model integrates the audio processing technology, the emotion analysis technology and the brain wave testing technology.

Further, the body-mind state indexes comprise five elements, yin-yang, color, five flavors, four natures, quality, state, orientation, sound dynamics, and audio-visual attributes of the human voice audios uploaded by the user.

Further, the modelling module comprises the following units:
a building unit of the Huiyin agent, configured to build a Huiyin agent based on the collected digital signals of all sounds in the natural environment, segment the human voice audios through the Huiyin agent, and assign tag data to each segment of the human voice audios;
a building unit of the Yunjing knowledge base, configured to build a Yunjing knowledge base based on tag data of the human voice audios, and manage the human voice audios through the Yunjing knowledge base; and
a building unit of the sound-assisted model, configured to build the sound-assisted model based on the Shenglan large-scale model, so as to provide basis for subsequent programming.

Further, the analysis and evaluation module comprises the following units:
an analysis unit, configured to analyze the human voice audios uploaded by the user based on the five-element theory of basic Chinese medicine and application of existing dialectical methods of traditional Chinese medicine, so as to obtain body-mind state indexes of the user; and
an AI evaluation unit, configured to continuously monitor and evaluate the physiological and psychological state of the user through AI technology, so as to obtain the evaluation result about the user.

Further, the holographic recording technology is adopted when sounds in a natural environment are sampled by the data acquisition module.

Further, after a personalized body-mind management program is built for the user, the programming module restores the digital signal of sounds.

Further, the programming module adopts the voiceprint holographic restoration technology when performing digital signal restoration of sounds.

Further, when building a field, the field building module performs targeted simulation based on required seasonal changes and specific needs of the user, and organically combines multi-dimensional elements including image, sound, smell and temperature according to needs of the user.

The field building module further combines the subjective participation in formulating drama performance according to needs of the user when building the field.

Further, the field building module adopts the holographic imaging technology when building a field.

The technical principles of the above solution are as follows:
The data acquisition module first collects the sounds in the natural environment and converts them into digital signals. Then, based on the digital signals, the modelling module builds a Huiyin agent to segment the human voice audios uploaded by the user and assign tag data to them, and builds a Yunjing knowledge base based on tag data to manage the human voice audios.

Moreover, the modelling module further builds a sound-assisted model based on the Shenglan large-scale model. Based on the five-element theory of traditional Chinese medicine, the programming module classifies the collected sounds in the natural environment through the sound-assisted model and analyzes the user's emotions and brain waves to obtain the specific situation of the user, perform permutation and combination of sounds according to the specific situation of the user, and build a personalized body-mind management program for the user.

During this period, the analysis and evaluation module will analyze the human voice audios uploaded by the user to obtain current body-mind state indexes of the user, and monitor and evaluate the body-mind states of the user through AI technology to obtain the evaluation result. The field building module will build different fields based on the user's body-mind management program.

The adoption of the above solution has the following beneficial effects:
1. The present disclosure selects sound materials in the natural environment dominated by natural sound sources, uses natural voiceprints to a great extent, and builds a natural ecological field that is more suitable for human beings, so that people who have lived in cities for a long time can get closer to nature, open their sensory systems, relax themselves, balance emotions, stimulate potential, and improve comprehensive field efficiency.
2. The present disclosure can build a personalized body-mind management program according to the specific situation of each user. This personalized management program can meet the needs of the user more accurately and improve the management effect.
3. The analysis and evaluation module in the present disclosure can comprehensively and deeply evaluate the user's physiological and psychological state based on the human voice audios uploaded by the user in combination with the five-element theory of traditional Chinese medicine and AI technology. The evaluation result can provide a scientific basis for the subsequent management program to ensure the pertinence and effectiveness of the program.
4. According to the present disclosure, in the process of data acquisition and restoration, the system adopts the holographic recording and the voiceprint holographic restoration technology, which ensures the accuracy and integrity of sound data. This efficient data processing technology can provide the user with a more true and accurate sound experience.
5. The present disclosure integrates a variety of technologies such as drama field, voiceprint and AI to form a brand-new body-mind management mode. This innovative technology fusion not only improves the practicality and operability of the system, but also brings a brand-new experience for the user' body-mind management.

Additional aspects and advantages of the present disclosure will be partially set forth in the following description, partially become apparent from the following description or be appreciated by practice of the present disclosure.

### BRIEF DESCRIPTION OF THE DRAWINGS

FIG. 1 is a schematic structural diagram of the body-mind management system based on drama field, voiceprint and AI technologies of the present disclosure.

FIG. 2 is a schematic structural diagram of the modelling module of the present disclosure.

FIG. 3 is a schematic structural diagram of the analysis and evaluation module of the present disclosure.

### DETAILED DESCRIPTION

The following are further described in detail with reference to specific embodiments:

### Embodiment 1

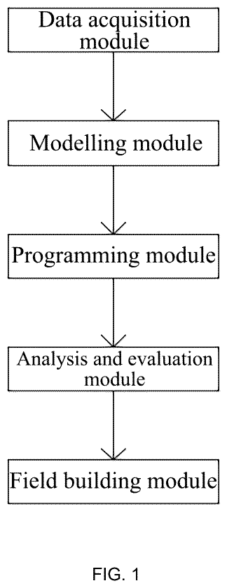

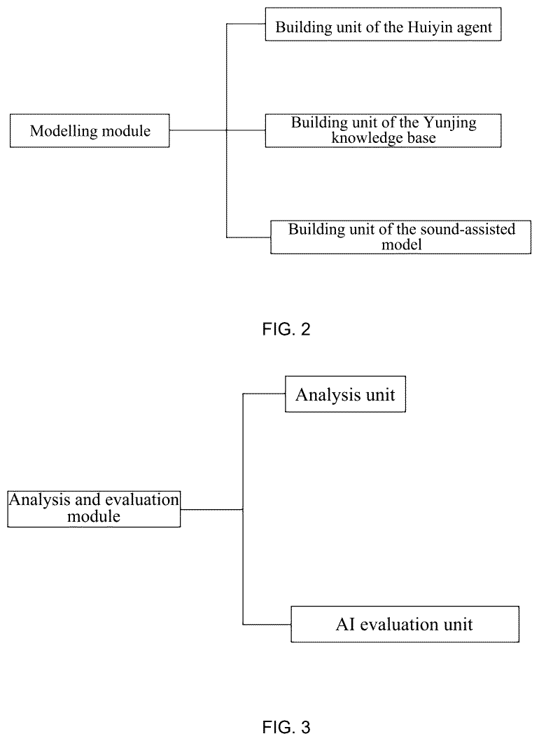

As shown in FIGS. 1, 2 and 3, a body-mind management system based on drama field, voiceprint and AI technologies comprising the following modules:
a data acquisition module, configured to sample sounds in the natural environment and convert them into digital signals;
a modelling module, configured to build a Huiyin agent based on the digital signals, segment the human voice audios uploaded by the user and assign tag data through the Huiyin agent, establish a Yunjing knowledge base based on the tag data, and manage the human voice audios;
the modelling module further builds a sound-assisted model that integrates the audio processing technology, the emotion analysis technology and the brain wave testing technology based on the Shenglan large-scale models;
the modelling module comprises the following units:
a building unit of the Huiyin agent, configured to build a Huiyin agent based on the collected digital signals of all sounds in the natural environment, segment the human voice audios through the Huiyin agent, and assign tag data to each segment of the human voice audios;
a building unit of the Yunjing knowledge base, configured to build a Yunjing knowledge base based on tag data of the human voice audios, and manage the human voice audios through the Yunjing knowledge base; and
a building unit of the sound-assisted model, configured to build the sound-assisted model based on the Shenglan large-scale model, so as to provide basis for subsequent programming.
a programming module, configured to classify the collected sounds in the natural environment based on the five-element theory of traditional Chinese medicine and the sound-assisted model, analyze the emotions and brain waves of the user to obtain the specific situation of the user, perform permutation and combination of sounds according to the specific situation of the user, and build a personalized body-mind management program for the user; after a personalized body-mind management program is built for the user, the programming module adopts the voiceprint holographic restoration technology to restore the digital signal of sounds;
an analysis and evaluation module, configured to analyze the human voice audios uploaded by the user to obtain current body-mind state indexes of the user, and monitor and evaluate the physiological and psychological states of the user through AI technology to obtain the evaluation result; and among them, the body-mind state indexes comprise five elements, yin-yang, color, five flavors, four natures, quality, state, orientation, sound dynamics, and audio-visual attributes of the human voice audios uploaded by the user;
the analysis and evaluation module comprises the following units:
an analysis unit, configured to analyze the human voice audios uploaded by the user based on the five-element theory of basic Chinese medicine and application of existing dialectical methods of traditional Chinese medicine, so as to obtain body-mind state indexes of the user;
an AI evaluation unit, configured to continuously monitor and evaluate the physiological and psychological state of the user through AI technology, so as to obtain the evaluation result about the user; and
a field building module, configured to build a specific drama field based on the evaluation result obtained by the analysis and evaluation module.

When building a field, the field building module uses the holographic imaging technology, performs targeted simulation based on required seasonal changes and specific needs of the user, and organically combines multi-dimensional elements including image, sound, smell and temperature according to needs of the user.

The field building module further combines the subjective participation in formulating drama performance according to needs of the user when building the field.

The specific implementation process is as follows: the data acquisition module first samples the sounds in the natural environment, quantizes and encodes them to convert them into digital signals. Then, the modelling module builds a Huiyin agent based on digital signals through the building unit of the Huiyin agent, performs segmentation operation on the human voice audios uploaded by the user through the Huiyin agent, and assigns tag data to each segment of the human voice audios. Then, the building unit of the Yunjing knowledge base builds a Yunjing knowledge base based on tag data of the human voice audios, and manage the human voice audios through the Yunjing knowledge base.

At this time, the analysis and evaluation module will analyze the human voice audios uploaded by the user through the analysis unit based on the five-element theory of basic traditional Chinese medicine and application of existing dialectical methods of traditional Chinese medicine, such as eight principal syndrome differentiation, six channel syndrome differentiation and triple energizer syndrome differentiation, to obtain the body-mind state indexes of the user. The AI evaluation unit will continuously monitor the body-mind state of the user through AI technology to obtain the evaluation result about the user.

The modelling module will further build a sound-assisted model based on the Shenglan large-scale model. After the sound-assisted model is built, the programming module classifies the collected sounds in the natural environment based on the five-element theory of traditional Chinese medicine and the sound-assisted model, analyzes the emotions and brain waves of the user to obtain the specific situation of the user, and builds a personalized body-mind management program for the user according to the specific situation of the user.

In this process, the field building module will perform targeted simulation based on required seasonal changes and specific needs of the user, and organically combine images, sounds, smells and temperature elements according to needs of the user, and use the holographic imaging technology to build different scene fields, including but not limited to educational cognition scenes, emotion regulation scenes and body-mind assisting scenes, so as to adapt to the dilemmas faced by different the user respectively. so as to serve different groups of people in the society more widely.

In this process, the field building module will further combine the subjective participation in formulating drama performance according to needs of the user when building the field.

For example, when the user needs to carry out talent cultivation, the field building module performs permutation and combination of sounds, images, temperatures, smells and seasons according to needs of the user to build a field that can be applied to educational cognitive scenes, providing the user with a field that is helpful for learning and improving his/her cognition; when the user needs to deal with the current emotion dilemma, the field building module can performs permutation and combination of sounds, images, temperatures, smells and seasons according to needs of the user to build a field that can be applied to the emotion regulation scene, thereby regulating the body-mind state of the user.

The present disclosure integrates a variety of technologies such as drama field, voiceprint and AI to form a brand-new body-mind management mode, which not only improves the practicability and operability of the system, but also brings a brand-new experience for the user's body-mind management. When evaluating the human voice audio uploaded by the user, the analysis and evaluation module combines the five-element theory of traditional Chinese medicine and AI technology to comprehensively and deeply evaluate the physiological and psychological state of the user, providing a scientific basis for subsequent management program to ensure the pertinence and effectiveness of the program.

### Embodiment 2

The difference from Embodiment 1 lies in that the holographic recording technology is adopted when sounds in a natural environment are sampled by the data acquisition module.

The specific implementation process is as follows: During the sampling operation, the holographic recording technology can completely record and reproduce all information related to sounds, including amplitude, frequency, phase, etc. of sounds, and even include vibration information generated by sounds. Compared with traditional recording methods, it can capture more sound details, making the recording results more true and complete.

Obviously, the above embodiments are merely examples for clarity of explanation and do not limit the implementation mode. For those of ordinary skills in the art, other different forms of changes or variations can be made on the basis of the above description. An exhaustive list of all embodiments is not necessary and cannot be given here. However, the obvious changes or variations arising therefrom are still within the protection scope of the present disclosure.

What is claimed is:

1. A body-mind management system based on drama field, voiceprint and AI technologies, comprising the following modules:
a data acquisition module, configured to sample sounds in the natural environment and convert them into digital signals;
a modelling module, configured to build a Huiyin agent based on the digital signals, segment the human voice audios uploaded by the user and assign tag data through the Huiyin agent, establish a Yunjing knowledge base based on the tag data, and manage the human voice audios;
the modelling module further builds a sound-assisted model based on the Shenglan large-scale model;
a programming module, configured to classify the collected sounds in the natural environment based on the five-element theory of traditional Chinese medicine and the sound-assisted model, analyze the emotions and brain waves of the user to obtain specific conditions of the user, perform permutation and combination of sounds according to the specific situation of the user, and build a personalized body-mind management program for the user;
an analysis and evaluation module, configured to analyze the human voice audios uploaded by the user to obtain current body-mind state indexes of the user, and monitor and evaluate the physiological and psychological states of the user through AI technology to obtain the evaluation result; and
a field building module, configured to build a specific drama field based on the evaluation result obtained by the analysis and evaluation module.

2. The body-mind management system based on drama field, voiceprint and AI technologies according to claim 1, wherein the sound-assisted model integrates the audio processing technology, the emotion analysis technology and the brain wave testing technology.

3. The body-mind management system based on drama field, voiceprint and AI technologies according to claim 2, wherein the body-mind state indexes comprise five elements, yin-yang, color, five flavors, four natures, quality, state, orientation, sound dynamics, and audio-visual attributes of the human voice audios uploaded by the user.

4. The body-mind management system based on drama field, voiceprint and AI technologies according to claim 3, wherein the modelling module comprises the following units:
a building unit of the Huiyin agent, configured to build a Huiyin agent based on the collected digital signals of all sounds in the natural environment, segment the human voice audios through the Huiyin agent, and assign tag data to each segment of the human voice audios;
a building unit of the Yunjing knowledge base, configured to build a Yunjing knowledge base based on tag data of the human voice audios, and manage the human voice audios through the Yunjing knowledge base; and
a building unit of the sound-assisted model, configured to build the sound-assisted model based on the Shenglan large-scale model, so as to provide basis for subsequent programming.

5. The body-mind management system based on drama field, voiceprint and AI technologies according to claim 4, wherein the analysis and evaluation module comprises the following units:
an analysis unit, configured to analyze the human voice audios uploaded by the user based on the five-element theory of basic Chinese medicine and application of existing dialectical methods of traditional Chinese medicine, so as to obtain body-mind state indexes of the user; and
an AI evaluation unit, configured to continuously monitor and evaluate the physiological and psychological state of the user through AI technology, so as to obtain the evaluation result about the user.

6. The body-mind management system based on drama field, voiceprint and AI technologies according to claim 5, wherein the holographic recording technology is adopted when sounds in a natural environment are sampled by the data acquisition module.

7. The body-mind management system based on drama field, voiceprint, and AI technologies according to claim 6, wherein after a personalized body-mind management program is built for the user, the programming module restores the digital signal of sounds.

8. The body-mind management system based on drama field, voiceprint and AI technologies according to claim 7, wherein the programming module adopts the voiceprint holographic restoration technology when performing digital signal restoration of sounds.

9. The body-mind management system based on drama field, voiceprint and AI technologies according to claim 8, wherein when building a field, the field building module performs targeted simulation based on required seasonal changes and specific needs of the user, and organically combines multi-dimensional elements including image, sound, smell and temperature according to needs of the user; and
the field building module further combines the subjective participation in formulating drama performance according to needs of the user when building a field.

10. The body-mind management system based on drama field, voiceprint and AI technologies according to claim 9, wherein the field building module adopts the holographic imaging technology when building a field.

11. A body-mind management device, wherein the body-mind management device is equipped with a body-mind management system based on drama field, voiceprint and AI technologies according to claim 1.

12. A computer-readable storage medium, wherein the computer-readable storage medium stores computer-readable instructions, and the computer-readable instructions implement a management method corresponding to the body-mind management system according to claim 1 when executed by a processor.
